(19) United States
(12) Patent Application Publication
(10) Pub. No.: US 2025/0354114 A1
(43) Pub. Date: Nov. 20, 2025

(54) METHODS OF PRODUCING ENGINEERED IMMUNE CELLS

(71) Applicant: Takeda Pharmaceutical Company Limited, Chuo-ku, Osaka-shi, Osaka (JP)

(72) Inventors: Soichiro Ogaki, Fujisawa-shi, Kanagawa (JP); Hideo Araki, Fujisawa-shi, Kanagawa (JP); Yoshiki Nakamura, Fujisawa-shi, Kanagawa (JP); Eiki Maeda, Fujisawa-shi, Kanagawa (JP)

(73) Assignee: Takeda Pharmaceutical Company Limited, Chuo-ku, Osaka-shi, Osaka (JP)

(21) Appl. No.: 18/866,855

(22) PCT Filed: May 19, 2023
(86) PCT No.: PCT/IB2023/055198

§ 371 Date: Nov. 18, 2024

Related U.S. Application Data
(60) Provisional application No. 63/344,255, filed on May 20, 2022.

(51) Int. Cl. C12N 5/0783; A61K 9/00; A61K 35/17; A61K 40/11; A61K 40/31; A61K 40/42; C12N 5/10; C12N 15/867

(52) U.S. Cl. CPC C12N 5/0636; A61K 9/0019; A61K 35/17; A61K 40/11; A61K 40/31; A61K 40/42; C12N 5/10; C12N 15/867; C12N 2510/00; C12N 2740/13043

(57) ABSTRACT

The present disclosure provides improved methods of producing engineered T cells that express an exogenous gene (e.g., CAR-T cells). T cells that express an exogenous gene and compositions comprising the same of the present technology are useful for treating various diseases, e.g., infection, autoimmune diseases, and tumors.

### CROSS-REFERENCE TO RELATED APPLICATIONS

The application claims the benefit of and priority to U.S. Provisional Patent Application No. 63/344,255, filed May 20, 2022, which is incorporated by reference herein in its entirety for any and all purposes.

### TECHNICAL FIELD

The present technology relates generally to improved methods of producing engineered immune cells that express an exogenous gene, including T cells expressing a chimeric antigen receptor (CAR-T cells).

### SUMMARY OF THE PRESENT TECHNOLOGY

In one aspect, the present disclosure provides a method of producing a population of T cells that express an exogenous gene product, the method comprising: (i) contacting a population of T cells with a stimulatory agent, (ii) contacting the population of T cells with a retroviral vector that comprises a nucleic acid molecule encoding the exogenous gene product, thereby providing a population of T cells that express the exogenous gene product, and (iii) collecting the population of T cells expressing the exogenous gene product for storage or administration, wherein: the population of T cells expressing the exogenous gene product from step (iii) are not expanded, or are expanded by no more than 200% as assessed by the number of living cells compared to the population of T cells at the beginning of step (i).

In some embodiments, the exogenous gene product is a chimeric antigen receptor (CAR).

In some embodiments, the stimulatory agent comprises a CD3 binding domain.

In some embodiments, the retroviral vector is a gamma retroviral vector. In some embodiments, the gamma retroviral vector is selected from a pMSGV vector, a pMSCV vector, a pSFG vector, or a derivative thereof.

In some embodiments, steps (i)-(iii) are performed in a single vessel.

In some embodiments, step (ii) is performed in the presence of a soluble additive of a cationic amphipathic peptide.

In some embodiments, step (ii) is not initiated until after completion of step (i).

In some embodiments, prior to step (i), the population of T cells is enriched for T cells that express CD3, CD4 and/or CD8

In some embodiments, step (i) is performed in about 4 to about 96 hours.

### BRIEF DESCRIPTION OF THE DRAWINGS

FIGS. 1A-1C show the comparison of a CAR-T cell manufacturing process when the activation step is skipped (Process #1), the transduction step is performed during the activation step (Process #2), and the activation step is 48 hours and followed by a 1-day transduction process (Process #3) (N=1). FIG. 1A shows the scheme of experimental design. FIGS. 1B and 1C show two experiments with Donor-3 and Donor-4 cells, showing the results of relative cell number after expansion (FIG. 1B, the cell number before expansion=1) and the percentage of CAR-expressing cells (FIG. 1C) obtained using a large-scale process.

FIGS. 2A-2C show the results of in vitro characterization during CAR-T cell manufacturing at Day 6 to Day 8 (N=1). As the number of expansion days increased, both proliferation capacity and pre-exhausted T cells, which include Stem Cell Memory T-cells and Central Memory T-cells, were decreased. FIG. 2A shows the scheme of experimental design. CAR-T cells were harvested at Day 6 to Day 8. After harvesting, CAR-T cells were formulated and cryopreserved. FIG. 2B shows the change in cell number during expansion (the cell number at day 3=1). FIG. 2C shows the result of proliferation capacity after thawing of cryopreserved Day 6 to Day 8 CAR-T cells. The proliferation capacity was measured by a “proliferation assay” (The cell number before proliferation assay=1). FIGS. 2D-2G show changes in the percentages of each T-cell phenotype of CAR-T cells from Day 6 to Day 8.

FIG. 3 shows the result of an in vivo efficacy study using Day 6, Day 7, and Day 8 CAR-T cells. CAR-T cells which were manufactured with a shorter expansion period showed superior anti-tumor efficacy in the HepG2-inoculated xenograft model. The X-axis represents the number of days after inoculation of a HepG2 cancer cell line. The Y-axis represents tumor volume in mice. Data are presented as mean±standard deviation, n=5.

FIGS. 4A-4I show the result of in vitro characterization of Day 3 and Day 7 CAR-T cells. FIG. 4A shows the scheme of a CAR-T cell manufacturing process. FIGS. 4B-4C (N=1) show the results of the percentage of CAR-expressing cells (FIG. 4B) and the copy number in the cells (FIG. 4C). FIGS. 4D-4E (N=1) show changes in T cell phenotype during manufacturing in CD4+ and CD8+ T cells, respectively. The legend in FIGS. 4D-4E is Tn=Naïve T cell, Tscm=Stem cell memory T cell, Tcm=Central memory T cell, Tem=Effector memory T cell and Teff=Effector T cell. FIGS. 4F-4G show fold increase after thawing of cryopreserved Day 3 and Day 7 CAR-T cells measured by an “expandability assay.” The number of CAR+CD4+ T cells (FIG. 4F) or CAR+CD8+ T cells (FIG. 4G) were determined by multiplying cell counts by nucleocounter with % CAR+, CD4+ and CD8+ by flow cytometry. FIG. 4H shows an assay scheme of a cytotoxicity assay with repeated antigen exposure. FIG. 4I shows luciferase activity in the live target cells over four days of culturing. The percentage of luciferase activity was determined using the ratio of luminescence of co-cultured wells as compared to tumor-only wells. Data are presented as mean±standard deviation, n=9 (Day1), 6 (Day2), 6 (Day3), or 3 (Day4). **p<0.01, n.s.; not significant, two-sided Student's t test.

FIGS. 5A and 5B show the results of an in vivo anti-tumor efficacy study using Day 3 and Day 7 CAR-T cells in a murine xenograft model. Luciferase-expressing GSU cells were subcutaneously inoculated into NSG mice. 2×10⁵ and 5×10⁵ cells of Day 3 CAR-T cells, 2×10⁵ and 1×10⁶ cells of Day 7 CAR-T cells, or PBS were intravenously administered to the mice. FIG. 5A shows a graph summarizing the in vivo data. The X-axis represents the number of days after CAR-T administration. The Y-axis represents tumor growth determined by bioluminescence in the mice. Data are presented as mean±standard deviation, n=3 or 5. Significance against a PBS-treated group at day 12 was analyzed using the Bartlett's test followed by the Dunnett's test (**p<0.01; ***p<0.001). FIG. 5B shows images of in vivo luminescence in the anti-tumor efficacy study.

FIGS. 6A and 6B show the results of an in vivo anti-tumor efficacy study using Day 3 and Day 7 CAR-T cells prepared with or without Prodigy. Luciferase-expressing GSU cells were subcutaneously inoculated into NSG mice. 5×10⁵ cells of Day 3 CAR-T cells, 1×10⁶ cells of Day 7 CAR-T cells, or PBS were intravenously administered to the mice. FIG. 6A shows a graph summarizing the in vivo data. The X-axis represents the number of days after CAR-T administration. The Y-axis represents tumor growth determined by bioluminescence in the mice. Data are presented as mean±standard deviation, n=3 or 5. Significance against a PBS-treated group at day 12 was analyzed using the Bartlett's test followed by the Dunnett's test (**p≤0.01; ***p≤0.001). FIG. 6B shows images of in vivo luminescence in the anti-tumor efficacy study.

FIGS. 7A-7F show the results of in vitro characterization of both Day 3 and Day 7 unarmored CAR-T cells (N=1). FIG. 7A shows the scheme of a CAR-T cell manufacturing process. FIGS. 7B-7C show the results of the percentage of CAR-expressing cells (FIG. 7B) and the copy number in the cells (FIG. 7C) using the scheme in FIG. 7A. FIGS. 7D-7E show changes in T cell phenotype during manufacturing in CD4+ and CD8+ T cells, respectively. The legend in FIGS. 7D-7E is Tn=Naïve T cell, Tscm=Stem cell memory T cell, Tcm=Central memory T cell, Tem=Effector memory T cell and Teff=Effector T cell. FIG. 7F shows the fold increase of cell number after 2 days culturing after thawing cryopreserved Day 3 and Day 7 CAR-T cells. The fold increase was calculated according to the ratio of cell counts 2 days after culturing as compared to the initial cell number, as determined by a nucleocounter.

FIG. 8 shows the result of an in vivo anti-tumor efficacy study using Day 3 and Day 7 unarmored CAR-T cells in a murine xenograft model. GSU cells were subcutaneously inoculated into NSG mice. Seven days after inoculation, 1×10⁵ of Day 3 and Day 7 CAR-T cells, or PBS were intravenously administered to the mice. The X-axis represents the number of days after CAR-T administration. The Y-axis represents tumor growth determined by a handheld Peira TM900 imaging device. Data are presented as mean±standard deviation, n=5. Significance among groups at day 42 was analyzed using the Bartlett's test followed by the Dunnett's test (n.s, not significant; ***p<0.001).

FIGS. 9A-9E show the results of in vitro characterization of both Day 3 and Day 7 CAR-T cells manufactured with CliniMACS Prodigy (N=1). FIG. 9A shows the scheme of the CAR-T cell manufacturing process. CAR-T cells were manufactured with CliniMACS Prodigy (Day 3 CAR-T cells). Day 3 CAR-T cells were further cultured in a G-Rex bottle for 4 days (Day 7 CAR-T cells). FIG. 9B-9C shows the results of percentage of CAR-expressing cells (FIG. 9B) and the copy number in the cells (FIG. 9C). FIGS. 9D-9E show changes in T cell phenotype during manufacturing in CD4+ and CD8+ T cells, respectively. The legend in FIGS. 9D-9E is Tn=Naïve T cell, Tscm=Stem cell memory T cell, Tcm is Central memory T cell, Tem=Effector memory T cell and Teff=Effector T cell.

FIG. 10 shows the result of the percentage of CAR-expressing cells manufactured with CliniMACS Prodigy with or without Vectofusin-1 in the transduction process (N=1).

### DETAILED DESCRIPTION

It is to be appreciated that certain aspects, modes, embodiments, variations, and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

In practicing the present methods, many conventional techniques in molecular biology, protein biochemistry, cell biology, microbiology and recombinant DNA are used. See, e.g., Sambrook and Russell eds. (2001) Molecular Cloning. A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture ofAnimal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present technology provides an improved method of producing engineered immune cells that express an exogenous gene for cell therapy, wherein the engineered immune cells include but are not limited to T cells expressing chimeric antigen receptor (CAR), T cells expressing T cell receptor (TCR), and T cells expressing synthetic T cell antigen receptor (STAR). In one embodiment, provided herein is a rapid CAR-T cell manufacturing process with in vivo CAR-T expansion, that is, a CAR-T cell manufacturing process requiring no or limited ex vivo CAR-T expansion (e.g., no more than 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% ex vivo expansion). In one embodiment, the improved CAR-T cell manufacturing process entails an activation step of about one to two days involving a stimulating agent such as anti-CD3 and anti-CD28 antibodies, and a transduction step of about 1 day (e.g., 20 to 28 hours) involving a retroviral vector that comprises a nucleic acid molecule encoding the CAR. In one embodiment, the manufacturing process of the present technology produces CAR-T cells exhibiting improved in vitro and in vivo expansion capacity. The resulting CAR-T cells also exhibited improved potency (in vivo anti-tumor effect) and in vivo proliferation. Additionally, in one embodiment, the manufacturing process of the present technology enables an all-in-one process, i.e., the manufacturing process may be performed in a single vessel in one embodiment.

### Definitions

Unless defined otherwise, all technical and scientific terms used herein have the meaning commonly understood by a person skilled in the art to which this disclosure belongs. The following references provide one of ordinary skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another.

As used herein, the term “activation” refers to the state of a T cell that has been sufficiently stimulated to induce cytokine production, detectable effector functions, and/or detectable cellular proliferation.

As used herein, the term “antibody” refers to an immunoglobulin molecule which specifically binds with an antigen. Antibodies may be intact immunoglobulins derived from natural sources or from recombinant sources and maybe be immunoreactive portions of intact immunoglobulins. The antibody in the present disclosure may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, “antibody fragment” or “antigen binding fragment” refers to Fab, Fab′, F(ab′)2, and Fv fragments, linear antibodies, sdAb (either V_{L} or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}. The term “linker” refers to synthetic sequences (e.g., amino acid sequences) that connect or link two sequences, e.g., that link two polypeptide domains. In some embodiments, the linker contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acid residues.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

The term “synthetic antibody” as used herein refers to an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present technology includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell, or a biological fluid.

The term “auto-antigen” means, in accordance with the present disclosure, any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

The term “autoimmune disease” as used herein is defined as a disorder that results from an autoimmune response. An autoimmune disease is the result of an inappropriate and excessive response to a self-antigen (auto-antigen). Examples of autoimmune diseases include but are not limited to, Addison's disease, alopecia greata, ankylosing spondylitis, autoimmune hepatitis, autoimmune parotitis, Celiac disease, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, autoimmune vasculitis, vitiligo, myxedema, pernicious anemia, ulcerative colitis, among others.

As used herein, the term “autologous” is meant to refer to any material derived from the same individual to which it is later to be re-introduced into the individual. “Allogeneic” refers to a graft derived from a different animal of the same species. “Xenogeneic” refers to a graft derived from an animal of a different species.

The term “tumor” or “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

“Co-stimulatory ligand,” as the term is used herein, includes a molecule on an antigen presenting cell (e.g., dendritic cell, B cell, macrophage, monocyte, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), B7-H1 (PD-L1), B7-DC (PD-L2), B7-H2, B7-H3, B7-H4, B7-H6, B7-H7/HHLA2, BTLA, 4-1BBL, OX40L, PDCD6, VISTA (B7-H5, PD-1H), GITRL (TNFSF18), inducible costimulatory ligand (ICOS-L), intercellular adhesion molecule (ICAM), CD27 Ligand (TNFSF7), CD28, CD28H (IGPR-1), CD30L, CD40, CD70, CD83, CTLA-4, HLA-G, MICA, MICB, HVEM, lymphotoxin beta receptor, 3/TR6, ILT3, ILT4, HVEM, TIM-1/KIM-1/HAVCR, TIM-4, Semaphorin 4A, Galectin-9, Butirophilins like BTN1A1 (Butyrophilin), BTN2A1, BTN2A2 (Butyrophilin 2A2), BTN3A1/2, BTN3A2, BTN3A3, BTNL2/Butyrophilin-like 2, BTNL3, BTNL4, BTNL6, BTNL8, BTNL9, BTNL10, CD277/BTN3A1, LAIR1, LAIR2, CD96, CD155/PVR, CRTAM, DNAM-1 (CD226), Nectin-2 (CD112), Nectin-3, PVRIG, TIGIT, LILRA3(CD85e), LILRA4 (CD85g, ILT7), LILRB3 (CD85a, ILT5), LILRB2 (CD85d, ILT4), LILRB1 (CD85j, ILT2), LILRB4 (CD85k, ILT3), B-cell-activating factor (BAFF) (BLyS, TNFSF13B), TL1A (TNFSF15), TNF-alpha, an agonist or antibody that binds Toll-like receptor (TLR) and a ligand that specifically binds with B7-H3. A co-stimulatory ligand also encompasses, inter alia, an antibody that specifically binds with a co-stimulatory molecule present on a T cell.

As used herein, the term “co-stimulatory molecule” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, CD40L, PD-1, PDL-1, ICOS (CD278), LFA-1, CD2, CD7, LIGHT, NKD2C, B7-H3, CTLA-4, GITR (TNFRSF18), TIM-1, TIM-2, TIM-3, TIM-4, CD160, CD200, CD300a (LMIR1), CD300d (LMIR4), CLECL1 (DCAL-1), DAP12, Dectin-1 (CLEC7A), DPPIV (CD26), EphB6, Integrin alpha 4 beta 1, Integrin alpha 4 beta 7/LPAM-1, LAG-3, TSLP R, B-cell-activating factor Receptor (BAFF R) (TNFRSF13C), DR3 (TNFRSF25), Lymphotoxin-alpha (TNF-beta), RELT (TNFRSF19L), TACI (TNFRSF13B), TNFR2 (TNFRSF1B), 2B4 (CD244, SLAMF4), BLAME (SLAMF8), CD2, CD2F-10 (SLAMF9), CD48 (SLAMF2), CD58 (LFA-3), CD84 (SLAMF5), CD229 (SLAMF3), CRACC (SLAMF7), NTB-A (SLAMF6), SLAM (CD150), and a ligand that specifically binds CD83. Accordingly, while the present disclosure provides exemplary costimulatory domains derived from CD28 and 4-1BB, other costimulatory domains are contemplated for use with the CARs described herein. The inclusion of one or more co-stimulatory signaling domains can enhance the efficacy and expansion of T cells expressing CAR receptors. The intracellular signaling and co-stimulatory signaling domains can be linked in any order in tandem to the carboxyl terminus of the transmembrane domain.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

As used herein “endogenous” refers to any material from or produced inside an organism, cell, tissue, or system.

As used herein, the term “exogenous” refers to any material introduced from or produced outside an organism, cell, tissue, or system.

The term “expression” as used herein is defined as the transcription and/or translation of a particular nucleotide sequence driven by its promoter.

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid may be from another organism, or it may be, for example, a mRNA molecule that is not normally expressed in a cell or sample.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The terms “substantially homologous” or “substantially identical” mean a polypeptide or nucleic acid molecule that exhibits at least 50% or greater homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or about 99% homologous or identical at the amino acid level or nucleic acid to the sequence used for comparison (e.g., a wild-type, or native, sequence). In some embodiments, a substantially homologous or substantially identical polypeptide contains one or more amino acid substitutions, insertions, or deletions relative to the sequence used for comparison. In some embodiments, a substantially homologous or substantially identical polypeptide contains one or more non-natural amino acids or amino acid analogs, including, D-amino acids and retroinverso amino, to replace homologous sequences.

As used herein, a “host cell” is a cell that is used to receive, maintain, reproduce, and amplify a vector. A host cell also can be used to express the polypeptide encoded by the vector. The nucleic acid contained in the vector is replicated when the host cell divides, thereby amplifying the nucleic acids.

As used herein, the term “immune cell” refers to any cell that plays a role in the immune response of a subject. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes. As used herein, the term “engineered immune cell” refers to an immune cell that is genetically modified. As used herein, the term “native immune cell” refers to an immune cell that naturally occurs in the immune system.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. As used herein, a “purified” or “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cells that have been separated from the cells with which they are naturally associated in their natural state. In some embodiments, the cells are cultured in vitro. In other embodiments, the cells are not cultured in vitro.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), intracisternal, intrathecal, or intrasternal injection, administration, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology such as PCR and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. A “constitutive” promoter is a nucleotide sequence which, when operably linked with a polynucleotide which encodes or specifies a gene product, causes the gene product to be produced in a cell under most or all physiological conditions of the cell. An “inducible” promoter is a nucleotide sequence which, when operably linked with a polynucleotide which encodes or specifies a gene product, causes the gene product to be produced in a cell substantially only when an inducer which corresponds to the promoter is present in the cell. A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

As used herein, “regulatory sequence” or “regulatory region” of a nucleic acid molecule means a cis-acting nucleotide sequence that influences expression, positively or negatively, of an operably linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration, gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more. Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the term “sample” refers to clinical samples obtained from a subject. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

As used herein, the term “secreted” in reference to a polypeptide means a polypeptide that is released from a cell via the secretory pathway through the endoplasmic reticulum, Golgi apparatus, and as a vesicle that transiently fuses at the cell plasma membrane, releasing the proteins outside of the cell. Small molecules, such as drugs, can also be secreted by diffusion through the membrane to the outside of cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. The terms “specific binding,” “specifically binds to,” or is “specific for” a particular molecule (e.g., an antigen), as used herein, can be exhibited, for example, by a molecule having a K_{d} for the molecule to which it binds to of about 10⁻⁴ M, 10⁻⁵ M, 10⁻⁶ M, 10⁻⁷ M, 10⁻⁸ M, 10⁻⁹, 10⁻¹⁰ M, 10⁻¹¹ M, or 10⁻¹² M.

As used herein, the term “stimulation” refers to a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGFβ, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand” or “a stimulatory agent” as used herein, means a ligand that when present on an antigen presenting cell (e.g., a dendritic cell, a B-cell, a macrophage, a monocyte, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory agents are well-known in the art and encompass, inter alia, an MHC Class I molecule loaded with a peptide, a CD3 binding domain (e.g., an anti-CD3 antibody), a CD28 binding domain (e.g., a superagonist anti-CD28 antibody), a CD2 binding domain (e.g., a superagonist anti-CD2 antibody), and Concanavalin A (ConA).

The term “therapeutically effective amount” refers to the amount of the subject compound that will elicit the biological or medical response of a tissue, system, or subject that is being sought by the researcher, veterinarian, medical doctor, or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “T cell” includes naïve T cells, memory T cells, activated T cells, anergic T cells, tolerant T cells, and antigen-specific T cells. For more specific examples, the T cells of the presently disclosed subject matter include but are not limited to, CD4⁺ T cells, CD8⁺ T cells, T helper cells, cytotoxic T cells, central memory T cells, stem cell-like memory T cells (stem cell memory T cells or stem-like memory T cells), effector memory T cells (e.g., TM cells and TEMRA cells,) regulatory T cells (also known as suppressor T cells), Natural killer T cells (NKT), Mucosal associated invariant T cells, ap T cells, double negative T cells, and γδ T cells. Cytotoxic T cells (CTL or killer T cells) are a subset of T lymphocytes capable of inducing the death of infected somatic or tumor cells. In certain embodiments, the CAR-expressing T cells express Foxp3 to achieve and maintain a T regulatory phenotype. In some embodiments, the CAR-T cells are any immune cells derived from pluripotent stem cells (e.g., induced pluripotent stem (iPS) cells).

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. Therapeutic effects of treatment include, without limitation, inhibiting recurrence of disease, alleviation of symptoms, diminishment of any direct or indirect pathological consequences of the disease, preventing metastases, decreasing the rate of disease progression, amelioration or palliation of the disease state, and remission or improved prognosis.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. A vector includes viral vectors. Viral vectors are engineered viruses that are operably linked to exogenous genes to transfer (as vehicles or shuttles) the exogenous genes into cells.

The viral vector of the present technology may be a retroviral vector. One advantage that retroviral vectors offer is their ability to transduce their single-stranded RNA genome into a double stranded DNA molecule that stably integrates into the target cell genome. Thus, retroviral vectors can be used to permanently modify the host cell nuclear genome.

The retroviral vector of the present technology may be derived from any member of the Retroviridae family, such as Spumavirus or Fomie virus (e.g., human and monkey virus), betaretrovirus (e.g. MMTV), gammaretrovirus (e.g. MLV), alpharetrovirus (e.g. ALV), delta retrovirus (e.g. BLV and HTLV-1), lentivirus (e.g. HIV 1), and epsilonretrovirus (e.g., WDSV, and WEHV1/2) or a derivative thereof.

Any methods known to those of skill in the art for the insertion of heterologous nucleic acid sequence into a vector (e.g., a retroviral vector) can be used to construct expression vectors containing a nucleic acid encoding any of the polypeptides provided herein.

### CAR-T Cells

CAR-T cells provided herein are T cells engineered to express at least one chimeric antigen receptor (CAR). CARs are engineered receptors comprising an extracellular and intracellular domain. The extracellular domain comprises an antigen binding moiety. In some embodiments, the extracellular domain also comprises a hinge domain. The intracellular domain or otherwise the cytoplasmic domain comprises, a CD3ζ chain and/or a costimulatory signaling region. The costimulatory signaling region refers to a portion of the CAR comprising the intracellular domain of a costimulatory molecule. Costimulatory molecules are cell surface molecules other than antigens receptors or their ligands that are required for an efficient response of lymphocytes to antigen.

Between the extracellular domain and the transmembrane domain of the CAR, or between the cytoplasmic domain and the transmembrane domain of the CAR, there may be incorporated a linker or spacer domain. As used herein, the term “spacer domain” generally means any oligo- or polypeptide that functions to link the transmembrane domain to either the extracellular domain or, the cytoplasmic domain in the polypeptide chain. A spacer domain may comprise up to 300 amino acids, preferably 10 to 100 amino acids and most preferably 25 to 50 amino acids.

### Antigen Binding Moiety.

The choice of an antigen binding moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen moiety domain in the CAR of the presently disclosed subject matter include those associated with viral, bacterial, and parasitic infections, autoimmune disease, and cancer cells.

In one embodiment, the CAR of the presently disclosed subject matter can be engineered to target a tumor antigen of interest by way of engineering a desired antigen binding moiety that specifically binds to an antigen on a tumor cell. Tumor antigens may be proteins that are produced by tumor cells that elicit an immune response, particularly T-cell mediated immune responses. The selection of the antigen binding moiety of the presently disclosed subject matter will depend on the particular type of cancer to be treated. Tumor antigens are well known in the art and include, for example, a glioma-associated antigen, carcinoembryonic antigen (CEA), .beta.-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, CA125, CA19-9, MUC-1, WT-1, glypican 3 (GPC3), and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the presently disclosed subject matter may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond, or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, cMet, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA19-9, CA 15-3\CA 27.29\BCAA, CA 195, CA 242, CA-50, CAM43, CD68P\1, CO-029, FGF-5, G250, Ga733\EpCAM, glypican 3 (GPC3), HTgp-175, M344, MA-50, mesothelin, MG7-Ag, MOV18, MUC-1, NB/70K, NY-CO-1, RCAS1, SDCCAG16, TA-90\Mac-2 binding protein\cyclophilin C-associated protein, TAAL6, TAG72, TLP, TPS, and WT-1. In one embodiment, the antigen binding moiety of the CAR targets an antigen that includes but is not limited to cMet, CD 19, CD20, CD22, ROR1, Mesothelin, CD33/IL3Ra, cMet, PSMA, Glycolipid F77, EGFRvIII, GD-2, MY-ESO-1 TCR, MAGE A3 TCR, and the like.

Depending on the desired antigen to be targeted, the CAR of the presently disclosed subject matter can be engineered to include the appropriate antigen bind moiety that is specific to the desired antigen target. For example, if CD19 is the desired antigen that is to be targeted, an antibody for CD19 can be used as the antigen bind moiety for incorporation into the CAR of the invention.

### Transmembrane Domain.

With respect to the transmembrane domain, the CAR can be designed to comprise a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e., comprise at least the transmembrane region(s) of) the α, β or ζ chain of the T-cell receptor, CD28, CD3ε, CD45, CD4, CD5, CD8, CD9, CD16, CD22, CD33, CD37, CD64, CD80, CD86, CD134, CD137, CD154, or from an immunoglobulin such as IgG4. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

### Cytoplasmic Domain.

The cytoplasmic domain or otherwise the intracellular signaling domain of the CAR of the presently disclosed subject matter is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR of the presently disclosed subject matter include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the presently disclosed subject matter include those derived from TCRζ, FcRγ, FcRβ, CD3γ, CD3δ, CD3ε, CD5, CD22, CD79a, CD79b, and CD66d. It is particularly preferred that cytoplasmic signaling molecule in the CAR of the presently disclosed subject matter comprises a cytoplasmic signaling sequence derived from CD3ζ.

In some embodiments, the cytoplasmic domain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

The cytoplasmic signaling sequences within the cytoplasmic signaling portion of the CAR of the presently disclosed subject matter may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain is designed to comprise the signaling domain of CD3ζ and the signaling domain of CD28. In another embodiment, the cytoplasmic domain is designed to comprise the signaling domain of CD3ζ and the signaling domain of 4-1BB. In yet another embodiment, the cytoplasmic domain is designed to comprise the signaling domain of CD3ζ and the signaling domain of CD28 and 4-1BB.

In the present disclosure, CAR-T cells may encompass any immune cells expressing a CAR.

The CAR-T cells of the present technology can be administered to a subject (e.g., a human subject) in need thereof for the treatment of any diseases, including but are not limited to infection, autoimmune diseases, or tumor.

### Methods of Producing Engineered Immune Cells of the Present Technology

In one aspect, the present disclosure provides a method of producing a population of T cells that express an exogenous gene product, the method comprising (i) contacting a population of T cells with a stimulatory agent, (ii) contacting the population of T cells with a retroviral vector that comprises a nucleic acid molecule encoding the exogenous gene product, thereby providing a population of T cells that express the exogenous gene product, and (iii) collecting the population of T cells expressing the exogenous gene product for storage or administration, wherein the population of T cells expressing the exogenous gene product from step (iii) are not expanded, or are expanded by no more than 200% as assessed by the number of living cells compared to the population of T cells at the beginning of step (i). In some embodiments, the exogenous gene product is a chimeric antigen receptor (CAR).

### Step (i): Activation

The population of T cells, prior to step (i), may be obtained from any source known in the art, including but are not limited to peripheral blood mononuclear cells, bone marrow, lymph node tissue, cord blood, thymus tissue, tissue from a site of infection, ascites, pleural effusion, spleen tissue, tumors, T cells derived from pluripotent stem cells, and T cells derived from direct differentiation. In certain embodiments of the present technology, any T cell lines available in the art, may be used. In certain embodiments of the present technology, T cells may be obtained from a unit of blood collected from a subject using various techniques known to the skilled artisan, e.g., apheresis. In some embodiments, the population of T cells, prior to step (i), may be isolated from peripheral blood lymphocytes by lysing the red blood cells and depleting the monocytes.

Procedures for separation include, but are not limited to, density gradient centrifugation (e.g., using PERCOLL® gradient); counterflow centrifugal elutriation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels, and Fluorescence-Activated Cell Sorting (FACS).

In some embodiments, a specific subpopulation of T cells, such as CD3⁺, CD28⁺, CD4⁺, CD8⁺, CD45RA⁺, and CD45RO⁺ T cells, may be further isolated by positive or negative selection techniques. In some embodiments, the population of T cells, prior to step (i), may be enriched for T cells that express CD3, CD4 and/or CD8. Those selection techniques are well-known to a skilled artisan in the art. For a non-limiting example, CD4⁺ cells may be enriched by negative selection by treating the mixture of cells with a monoclonal antibody cocktail including antibodies to CD14, CD20, CD 11b, CD 16, HLA-DR, and CD8. In certain embodiments, regulatory T cells may be depleted by anti-CD25 conjugated beads.

In some embodiments, T cells, prior to step (i), may be frozen after a washing step or may be frozen without wash step. The freeze and subsequent thaw step may provide a more uniform product by removing granulocytes and to some extent monocytes in the cell population. After the washing step that removes plasma and platelets, the cells may be suspended in a freezing solution. The freezing solutions and parameters are known in the art. In certain embodiments, cryopreserved cells may be thawed and washed and allowed to rest for about an hour at room temperature prior to step (i).

The source of the T cells may be collected at any time point necessary for later activation, transduction, formulation, and for use in T cell therapy for any diseases or conditions that would benefit from T cell therapy. In one embodiment, a blood sample or an apheresis may be taken from a generally healthy subject. In certain embodiments, a blood sample or an apheresis may be taken from a generally healthy subject who is at risk of developing a disease, but who has not yet developed a disease, and the cells of interest are isolated and frozen for later use. In certain embodiments, samples may be collected from a patient shortly after diagnosis of a particular disease as described herein but prior to any treatments. In a further embodiment, the cells may be isolated from a blood sample or an apheresis from a subject prior to, during, or following any relevant treatment modalities, including but are not limited to treatment with agents such as antiviral agents, chemotherapy, radiation, immunotherapies (e.g., checkpoint inhibitors), or immunosuppressive agents.

In some embodiment of the present technology, T cells may be obtained from a patient directly following a treatment. In this regard, it has been observed that following certain cancer treatments, in particular treatments with drugs that damage the immune system, shortly after treatment during the period when patients would normally be recovering from the treatment, the quality of T cells obtained may be optimal or improved for ex vivo manipulation (e.g., activation and engineering).

In any of the above embodiments, the T cells are activated or stimulated by a stimulatory agent. The stimulatory agent may comprise an agent that stimulates a CD3/TCR complex associated signal and/or a ligand that stimulates a co-stimulatory molecule on the surface of the T cells. In some embodiments, the stimulatory agent comprises a CD3 binding domain, a CD28 binding domain, a CD134 binding domain, and/or a CD137 binding domain. In some embodiments, the stimulatory agent comprises a CD3 binding domain and/or a CD28 binding domain. In some embodiments, the stimulatory agent comprises an anti-CD3 antibody and/or an anti-CD28 antibody. Examples of anti-CD28 antibodies include but are not limited to 9.3, B-T3, and XR-CD28 (Diaclone, Besancon, France). Examples of anti-CD3 antibodies include but are not limited to OKT3, 145-2C11, 17A2, UCHT1, and SK7.

In certain embodiments, the stimulatory agent comprises an anti-CD3 antibody and an anti-CD28 antibody. Each of the anti-CD3 antibody and anti-CD28 antibody may be independently in solution or coupled to a surface. When both are coupled to a surface, the anti-CD3 antibody and anti-CD28 may be coupled to the same surface (i.e., in “cis” formation) or to separate surfaces (i.e., in “trans” formation).

In one embodiment, the anti-CD3 antibody and anti-CD28 antibody are immobilized on beads, either on the same bead, i.e., “cis,” or to separate beads, i.e., “trans.” In some embodiments, the mole ratio of anti-CD3 antibody to anti-CD28 antibody ranges from 100:1 to 1:100 and all integer values there between. In some embodiments, ratios of beads to cells may range from 1:500 to 500:1 and any integer values in between. Optimal ratios will vary depending on particle size and on cell size and type. In some embodiments, the stimulatory agent is MACS GMP T-Cell TransACT (Miltenyi Biotec) (“TransAct”). In some embodiments, the ratio of T cell suspension and TransAct is about 17:1. Those of ordinary skill in the art can readily appreciate that any cell concentration may be used. For example, in one embodiment, a concentration of about 10 to 15 million cells/ml, about 15 to 20 million cells/ml, about 20 to 25 million cells/ml, about 25 to 30 million cells/ml, about 30 to 35 million cells/ml, about 35 to 40 million cells/ml, about 40 to 45 million cells/ml, about 45 to 50 million cells/ml, about 50 to 55 million cells/ml, about 55 to 60 million cells/ml, about 60 to 65 million cells/ml, about 65 to 70 million cells/ml, about 70 to 75 million cells/ml, about 75 to 80 million cells/ml, about 80 to 85 million cells/ml, about 85 to 90 million cells/ml, about 90 to 95 million cells/ml, about 95 to 100 million cells/ml, about 100 to 125 million cells/ml, about 125 to 150 million cells/ml, about 150 to 200 million cells/ml, about 200 to 500 million cells/ml, about 500 million cells/ml to 1 billion cells/ml, or about 1 billion cells/ml to 2 billion cells/ml may be used.

In certain embodiments, it may be desirable to significantly increase the concentration of cells to ensure maximum contact of cells and particles. In addition, use of high cell concentrations allows more efficient capture of cells that may weakly express target antigens of interest. For example, using high concentration of cells allows more efficient selection of CD8+ T cells that normally have weaker CD28 expression.

In other embodiments, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4⁺ T cells expressing higher levels of CD28 may be more efficiently captured than CD8⁺ T cells in dilute concentrations.

In one embodiment, the T cells may be in contact with the stimulatory agent (e.g., anti-CD3 antibody and anti-CD28 antibody) for about 4 to about 96 hours, e.g, about 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, 72 hours, 73 hours, 74 hours, 75 hours, 76 hours, 77 hours, 78 hours, 79 hours, 80 hours, 81 hours, 82 hours, 83 hours, 84 hours, 85 hours, 86 hours, 87 hours, 88 hours, 89 hours, 90 hours, 91 hours, 92 hours, 93 hours, 94 hours, 95 hours, or about 96 hours. In some embodiments, the T cells may be in contact with the stimulatory agent for about 4 to 48 hours. In some embodiments, the T cells may be in contact with the stimulatory agent for about 12 to 48 hours. In some embodiments, the T cells may be in contact with the stimulatory agent for about 24 to 48 hours. The beads and the cells may be subsequently separated, and then the cells may be washed and collected for transduction.

### Step (ii): Transduction

In any embodiment, step (ii) (contacting the population of T cells with the retroviral vector), is not initiated until after completion of step (i) (contacting a population of T cells with a stimulatory agent). For example, the T cells from step (i) may be washed and collected for step (ii).

For the initial genetic modification of T cells to produce T cells expressing the exogenous gene product (e.g., CAR) in step (ii), a retroviral vector is generally employed for transduction. For example, a polynucleotide encoding a CAR can be cloned into a retroviral vector and expression can be driven from its endogenous promoter, from the retroviral long terminal repeat, or from an alternative internal promoter. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al., Mol. Cell. Biol. 5:431-437 (1985)); PA317 (Miller, et al., Mol. Cell. Biol. 6:2895-2902 (1986)); and CRIP (Danos, et al. Proc. Natl. Acad. Sci. USA 85:6460-6464 (1988)). Non-amphotropic particles are suitable too, e.g., particles pseudotyped with VSVG, RD 114 or GALV envelope and any other known in the art.

Possible methods of transduction also include direct co-culture of the T cells with producer cells, e.g., by the method of Bregni, et al., Blood 80: 1418-1422(1992), or culturing with viral supernatant alone or concentrated vector stocks with or without appropriate growth factors and polycations, e.g., by the method of Xu, et al., Exp. Hemat. 22:223-230 (1994); and Hughes, et al., J. Clin. Invest. 89: 1817 (1992). In some embodiments, contacting the population of T cells with a retroviral vector that comprises a nucleic acid molecule encoding the CAR is performed in the presence of a soluble additive of a cationic amphipathic peptide, e.g., Vectofusin-1. In some embodiments, contacting the population of T cells with a retroviral vector that comprises a nucleic acid molecule encoding the CAR is performed in the presence of fibronectin-like peptide (e.g. Retronectin).

In some embodiments, the retroviral vector expressing a presently disclosed CAR may be an oncoretroviral vector, a gammaretroviral vector, or a spumaretroviral vector. In some embodiments, the retroviral vector may be a gammaretroviral vector. In some embodiments, the gamma retroviral vector is selected from a pMSGV vector, a pMSCV vector, a pSFG vector, or a derivative thereof.

Retroviral vectors are particularly well developed and have been used in clinical settings (Rosenberg et al., N. Engl. J. Med 323:370 (1990); Anderson et al., U.S. Pat. No. 5,399,346). Other viral vectors that may be used include, for example, adenoviral, and adeno-associated viral vectors, vaccinia virus, a bovine papilloma virus, or a herpes virus, such as Epstein-Barr Virus.

In one embodiment, contacting the population of T cells with a retroviral vector that comprises a nucleic acid molecule encoding an exogenous gene product (e.g., CAR), may be performed for about 1 to about 72 hours, e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, or about 72 hours. In some embodiments, the T cells may be in contact with the retroviral vector that comprises a nucleic acid molecule encoding the CAR for about 1 to 28 hours. In some embodiments, the T cells may be in contact with the retroviral vector that comprises a nucleic acid molecule encoding an exogenous gene product (e.g., CAR) for about 16 to 28 hours, e.g., 24 hours.

Conditions appropriate for T cell culture in step (i) and/or (ii) include an appropriate media (e.g., Minimal Essential Media or RPMI Media 1640 or, X-vivo 15, (Lonza)) that may contain factors necessary for viability and/or proliferation, including but are not limited to serum (e.g., fetal bovine or human serum), interleukin-2 (IL-2), insulin, IFNγ, IL-4, IL-7, GM-CSF, IL-10, IL-12, IL-15, IL-21, TGFβ, and TNFα, or any other additives for the growth of cells known to the skilled artisan. Other additives for the growth of cells include, but are not limited to, surfactant, plasmanate, and reducing agents such as N-acetyl-cysteine and 2-mercaptoethanol. Media may include RPMI 1640, AIM-V, DMEM, MEM, α-MEM, F-12, IMDM, Advanced DMEM/F12, X-Vivo 10™, X-Vivo 15™, X-Vivo 20™, TheraPEAK™ X-Vivo 10, TheraPEAK™ X-Vivo 15™, TheraPEAK™ X-Vivo 20™, CTS™ Optimizer™ T Cell Expansion SFM, CTS Optmizer Pro Serum Free Medium, 4Cell Nutri-T Medium, LymphoONE™ T-Cell Expansion Xeno-Free Medium, ImmunoCult™-XF T Cell Expansion Medium, ExCellerate Human T Cell Expansion Medium, Stemline T Cell Expansion Medium, CAR T-Cell Medium, TexMACS™ Medium, Corning Lymphocyte Serum-free Medium, Corning 88-581-CM Medium, CellGenix T Cell Medium, SmarT™ T cell Expansion Medium, StemSpan™ Serum-Free Expansion Medium, and OptiPEAK T Lymphocyte XPR with added amino acids, sodium pyruvate, and vitamins, either serum-free or supplemented with an appropriate amount of serum (or plasma) or a defined set of hormones, and/or an amount of cytokine(s) sufficient for the growth and/or expansion of T cells. The target cells are maintained under conditions necessary to support growth, for example, an appropriate temperature (e.g., room temperature or 37° C.) and atmosphere (e.g., air plus 5% CO₂).

Step (iii): Storage, Formulation, and/or Administration

The engineered T cells expressing an exogenous gene product (e.g., CAR-T cells) from step (ii) are optionally washed and collected for step (iii) for storage, formulation, and/or administration, according to protocols well-known in the arts. In particular, the present technology of producing the engineered T cells (e.g., CAR-T cells) requires no or limited ex vivo expansion. In some embodiments, the population of the engineered T cells (e.g., CAR-T cells) from step (iii) are not expanded, or are expanded by no more than 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% as assessed by the number of living cells compared to the population of T cells at the beginning of step (i). Any methods of assessing living cell numbers known in the art may be used, including but are not limited to hemocytometer, Automated Cell Counters, and any known cell viability and cytotoxicity assays. Those methods may be based on PCR, Spectrophomete, pH meter, ATP measurement, weight measurement, or metabolite analyzer.

In some embodiments, steps (i)-(iii), are all performed in a single vessel. Suitable vessels for the method of producing the engineered T cells (e.g., CAR-T cells) of the present technology include but are not limited to G-REX® bioreactors, CliniMACS Prodigy®, Xuri™ Cell Expansion system, WAVE Bioreactor™, Coccon® platform, PBS bioreactor, Ambr bioreactor, Biostat bioreactor, Cell Factory systems, CellSTAK and Cell culture bag, Dish, Well plate, and Flask.

The engineered T cells (e.g., CAR-T cells) may be formulated for administration or for long term storage. For long term storage, the engineered T cells (e.g., CAR-T cells) from step (ii) may be cryopreserved. Methods for cryopreservation is well-known to a skilled in the art. For example, the engineered T cells (e.g., CAR-T cells) may be suspended in a cell cryopreservation solution containing cryoprotective agents (e.g., DMSO) and protein (e.g. human serum albumin), polymer and/or sugar, and subject to freezing at −80° C. for 1 day; cryopreserved cells were further stored in liquid nitrogen (LN) (e.g., <−150° C.). Many factors in cryopreservation affect the quality of the engineered T cells (e.g., CAR-T cells) thus the outcome of the cell therapy. Those factors include (1) formulation and introduction of a freezing medium, (2) cooling rate, (3) storage conditions, (4) thawing conditions, and (5) post-thaw processing. Optimization of those factors to achieve the desired outcome of a cell therapy is within the level of a person of ordinal skill in the art.

### Formulations

The engineered T cells (e.g., CAR-T cells) and compositions comprising the same of the present technology can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with engineered T cells (e.g., CAR-T cells) of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel, or another liquid form, such as a time release form or liquid-filled form).

Those skilled in the art will recognize that the components of the compositions should be selected to be chemically inert and will not affect the viability or efficacy of the engineered T cells (e.g., CAR-T cells) as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the engineered T cells (e.g., CAR-T cells) of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ the engineered T cells (e.g., CAR-T cells) of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more the engineered T cells (e.g., CAR-T cells) of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, engineered T cells (e.g., CAR-T cells) are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt. % to about 5 wt. %, from about 0.0001 wt. % to about 1 wt. %, from about 0.0001 wt. % to about 0.05 wt. %, from about 0.001 wt. % to about 20 wt. %, from about 0.01 wt. % to about 10 wt. %, or from about 0.05 wt. % to about 5 wt. %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And the time for sequential administrations can be ascertained without undue experimentation.

### Administration

The engineered T cells (e.g., CAR-T cells) of the presently disclosed subject matter can be provided systemically or directly to a subject for treating an infection, autoimmune diseases, or tumor. In certain embodiments, the engineered T cells (e.g., CAR-T cells) are directly injected into an organ of interest. Additionally or alternatively, the engineered T cells (e.g., CAR-T cells) are provided indirectly to the organ of interest, for example, by administration into the circulatory system or into the tissue of interest. Expansion and differentiation agents can be provided prior to, during or after administration of cells and compositions to increase production of T cells in vitro or in vivo.

The engineered T cells (e.g., CAR-T cells) of the presently disclosed subject matter can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising the engineered T cells (e.g., CAR-T cells) can comprise a purified population of cells. Those skilled in the art can readily determine the percentage of the engineered T cells (e.g., CAR-T cells) in a cell population using various well-known methods, such as fluorescence activated cell sorting (FACS). The ranges of purity in cell populations comprising the engineered T cells (e.g., CAR-T cells) can be from about 50% to about 55%, from about 55% to about 60%, about 60% to about 65%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The engineered T cells (e.g., CAR-T cells) can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising the engineered T cells (e.g., CAR-T cells) and a pharmaceutically acceptable carrier. Administration can be autologous or non-autologous (allogeneic). For example, the engineered T cells (e.g., CAR-T cells) and compositions comprising the same can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter, it can be formulated in a unit dosage injectable form (solution, suspension, emulsion).

Some embodiments of the present disclosure are further described below through examples. The examples are intended to be illustrative and are not meant in any way to limit the scope of the disclosure. Accordingly, the present disclosure is not limited to the specific embodiments described through examples herein.

### EXAMPLES

### General Experimental Methods

### Medium

CAR-T cell culture medium: 2.6% OpTmizer Expansion Basal Supplement (Thermo Fisher Scientific), 1% L-Glutamine (Thermo Fisher Scientific), and 1% Streptomycine, 2% CTS Immune Cell SR (Thermo Fisher Scientific) were added to OpTmizer CTS T-Cell Expansion basal medium (Thermo Fisher Scientific) to prepare a basal cell culture medium. To this basal cell culture medium was added 20 IU/mL or 40 IU/mL of MACS GMP IL-2 (Miltenyi Biotec).

SK-HEP-1-Luc and HepG2 cell culture medium: MEM, L-Gln (+) (Thermo Fisher Scientific) was prepared by adding 10% FBS (Biosera Co., Ltd.), 1% Non-essential amino acids (Fujifilm Wako Pure Chemical Industries, Ltd.), 1% Penicillin-Streptomycin solution (Fujifilm Wako Pure Chemical Industries, Ltd.), and 1 mM Sodium pyruvate (Fujifilm Wako Pure Chemical Industries, Ltd.).

GSU-Luc cell culture medium: RPMI1640 (Thermo Fisher Scientific) was prepared by adding 10% FBS (Biosera Co., Ltd.) and 1% Penicillin-Streptomycin solution (Fujifilm Wako Pure Chemical Industries, Ltd.).

### Production of CAR-T Cells

After Leukopak (Hemacare) was thawed, the CD4+ and CD8+ cells were enriched by CliniMACS Prodigy (Miltenyi Biotech). Enriched cells were diluted in CAR-T cell culture medium to less than or equal to 2.0×10⁶ cells/mL (pre-production raw material). Cell suspension: MACS GMP T-Cell TransACT (Miltenyi Biotec)=17.5:1 was seeded in culture bags and cultured in about 48 hours (cell activation step). The activated cells were diluted in culture medium using a LOVO Cell processing system (Fresenius Kabi) or a centrifuge, seeded under 6.07×10⁵ cells/cm² in culture bags that had been previously coated with Retronectin® (Takara Bio Co., Ltd.) and a retrovirus into which a CAR gene or a CAR gene, an IL-7 gene, and a CCL19 gene had been introduced, and cultured until the next day (gene transduction process). Culture bottles (G-Rex, Wilson Wolf) were seeded under 2.2×10⁶ cells/cm² and optionally cultured for 3-7 days to produce CAR-T cells. After cultivation, CAR-T cells were cryopreserved in Cryostor CS-10 Freezing Media (BioLife Solutions) as final products. The CAR gene used has the base sequence encoding the amino acid sequence shown in SEQ ID NO: 1 or SEQ ID NO:2, and IL-7 gene used has the base sequence encoding the amino acid sequence shown in SEQ ID NO: 3, and CCL19 gene used has the base sequence encoding the amino acid sequence shown in SEQ ID NO: 4. See Table 1.

Production of CAR-T Cells with CliniMACS Prodigy

After Leukopak (Hemacare) was thawed, the CD4+ and CD8+ cells were enriched in CliniMACS Prodigy (Miltenyi Biotech). After enrichment, cells were diluted in CAR-T cell culture medium with MACS GMP T-Cell TransACT (Miltenyi Biotech) and cultured for about 48 hours, followed by retrovirus transduction with or without MCAS GMP Vectofusin-1 (Miltenyi Biotech) which was conducted in CliniMACS Prodigy for 1 day. At day 3, transduced cells were recovered from CliniMACS Prodigy, then culture bottles (G-Rex, Wilson Wolf) were seeded under 2.2×10⁶ cells/cm² and optionally cultured for 3-7 days to produce CAR-T cells (final products). After cultivation, CAR-T cells were cryopreserved in CryoStor CS10 Freezing Media (BioLife Solutions) as final products.

### Determination of Transduction Rate and Immune Phenotype of T Cells Using Flow Cytometry

CAR introduction rates into T cells were determined using CAR-targeting antigens on a BD FACSCanto II flow cytometer (BD Biosciences). The immunophenotype of T cells was measured using an anti-CD4 antibody (clone SK3, cat #344604, BioLegend), an anti-CD8 antibody (clone SK1, cat #344710, BioLegend), an anti-CCR7 antibody (clone G043H7, cat #353204, BioLegend), an anti-CD45RA antibody (clone L48, cat #337167, BD Biosciences), an anti-CD27 antibody (clone 0323, cat #302836, BioLegend), and an anti-CD95 antibody (clone DX2, cat #305612, BioLegend). CCR7/CD45RA negative cells were effector memory T-cells, CCR7 positive CD45RA negative cells were central memory T-cells, CCR7 negative CD45RA positive cells were effector T cells, CCR7/CD45RA/CD27/CD95 positive cells were defined as stem cell memory T cells, and CCR7/CD45RA positive cells other than them were defined as naive T cells.

### Proliferation Assay

Frozen CAR-T cells were thawed and cultured in CAR-T cell culture medium for 3 days. After culture, cell counting was conducted.

### Expandability Assay

Frozen CAR-T cells were thawed and cultured in CAR-T cell culture medium without IL-2 for 7 days. After culture, cell counting was conducted.

### Cytotoxicity Assay

CAR-T cells (Effector cells) and luciferase-expressing SK-HEP-1 cells were seeded in cell culture plates with SK-HFP-1 culture medium at the ratio of Effector:Target=5:1 (0.1M:20K per well). After 24 hours incubation, effector cells were collected and counted by NC-200 instrument. In addition, luciferase activity derived from the target cells were measured to determine the target cell killing rate. All collected effector cells were transferred to new cell culture plates. 20K cells/well of target cells were reseeded to new plates before seeding of the effector cells. After 24 h from the reseeding, cell number and luciferase activity were remeasured. These steps were repeated at 24 h, 48 h, 72 h, 96 h and effector cell number and cytotoxicity activity were measured.

### In Vivo Experiment

GSU-Luc cells or HepG2 were subcutaneously inoculated into NSG mice (Charles River Japan). Seven days after inoculation, CAR-T cells or PBS were intravenously administered to the mice. To analyze tumor growth by measurement of luciferase activity of GSU-Luc cells, D-Luciferin (Promega) was intraperitoneally injected, followed by the measurement luminescence by IVIS imaging (Summit Pharmaceutical International). To determine the tumor volume of HepG2, calipers were used.

### Example 1: Activation Process is Essential for CAR-T Cells Manufacturing

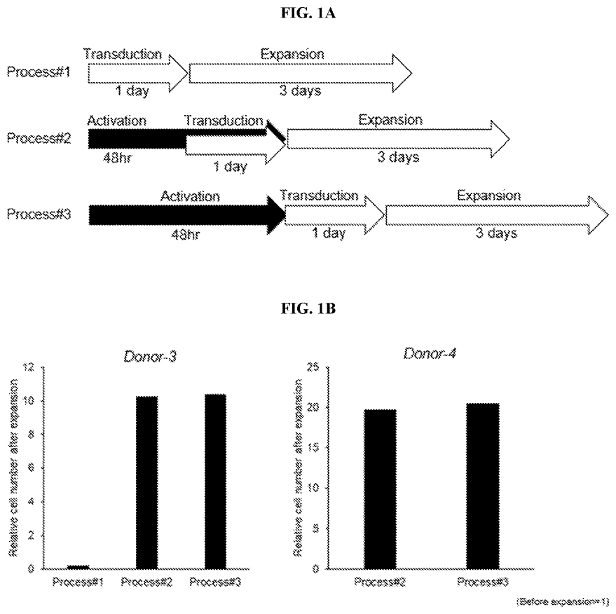

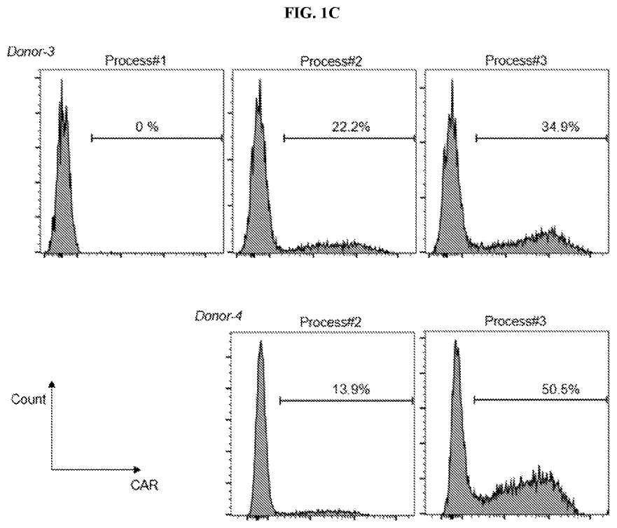

CAR-T cells expressing IL-7 gene and CCL19 gene were produced as described above (CAR described as SEQ ID NO:1 was used). It was compared that CAR-T manufacturing process when skipped activation step (Process #1), transduction step is performed during the activation step (Process #2), and 48-hour activation followed by 1-day transduction process (process #3) in two experiments, with Donor-3 and Donor-4, respectively (FIG. 1A). CAR-T cells which were manufactured as Process #1 could not be produced at all. However, CAR-T cells manufactured by both Process #2 and Process #3 can be produced without any issue (FIG. 1B and FIG. 1C).

### Example 2: In Vitro Characterization of CAR-T Cells During Expansion Step

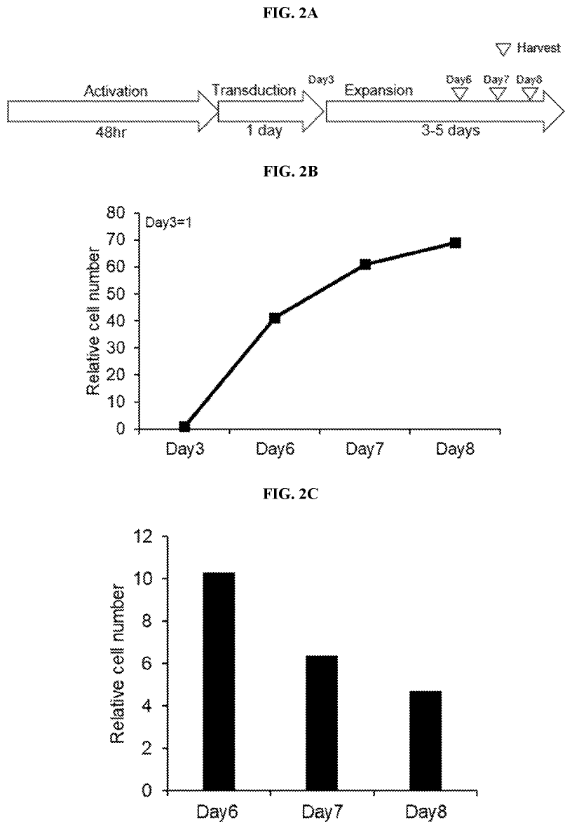

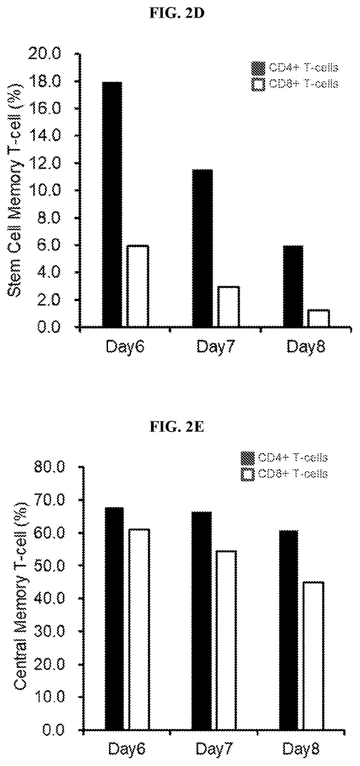

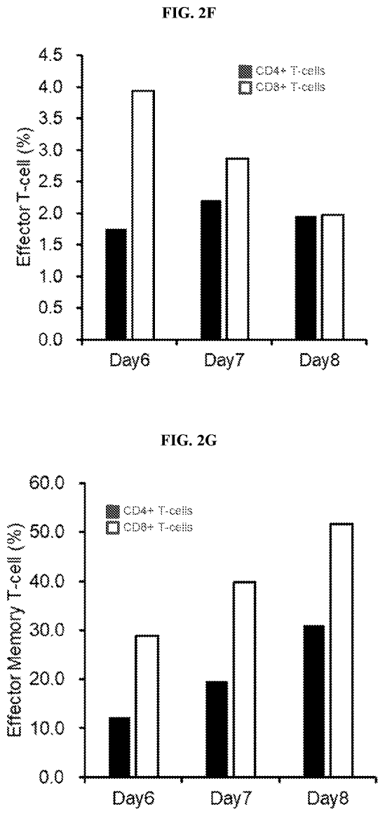

CAR-T cells expressing IL-7 gene and CCL19 gene were produced as described above (CAR described as SEQ ID NO: 1 was used). CAR-T cells were harvested at Day 6 to Day 8 (FIG. 2A). Cell number was increased day by day during manufacturing (FIG. 2B). Cell proliferation capacity and T cell phenotype were analyzed using Day 6 to Day 8 CAR-T cells (FIGS. 2C-2G). As expansion day goes on, both proliferation capacity and pre-exhausted cells, which include Stem Cell Memory T cells and Central Memory T cells, are decreased. These results suggested that changes in the cell quality including both proliferation capacity and percentages of each T cell phenotype of CAR-T cells from Day 6 to Day 8.

### Example 3: In Vivo Study with Day 6 to Day 8 CAR-T Cells

CAR-T cells produced in Example 2 were administrated after 7 days of HepG2 inoculation into NSG mice. CAR-T cells which are manufactured with shorter expansion period show superior anti-tumor efficacy in the HepG2-inoculated xenograft model (FIG. 3).

### Example 4: In Vitro Characterization of Day 3 and Day 7 CAR-T Cells

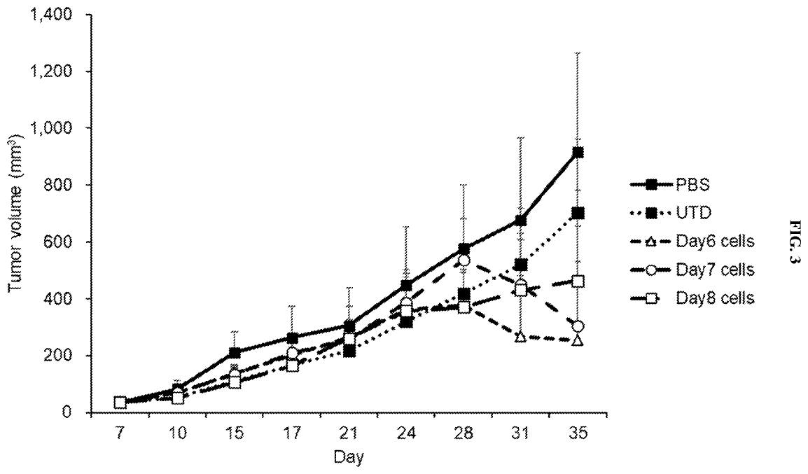

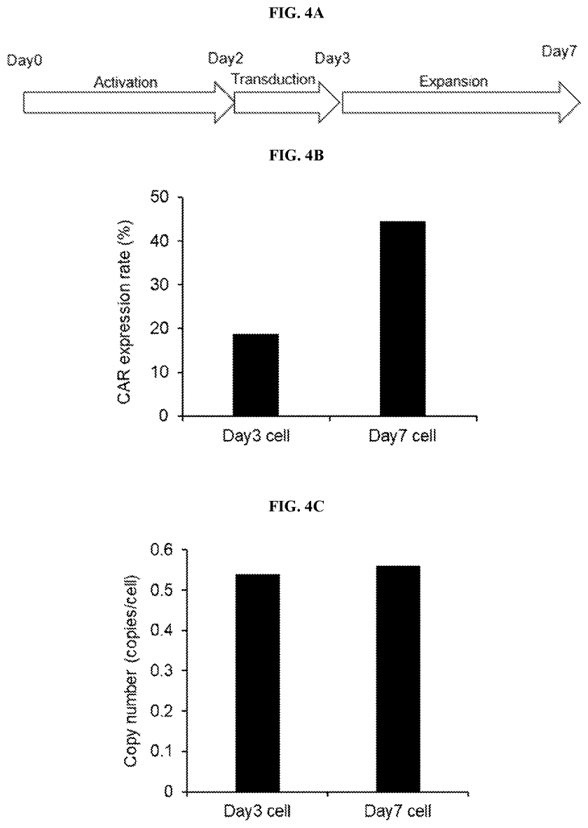

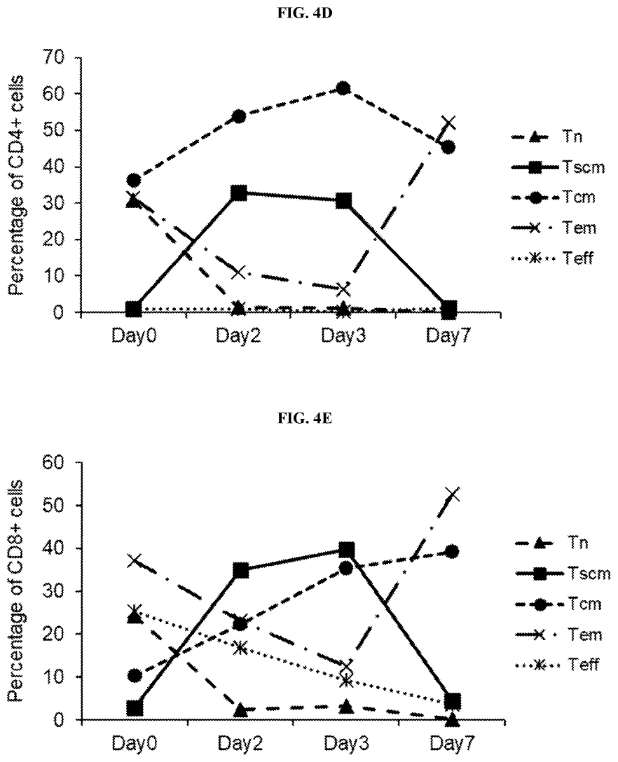

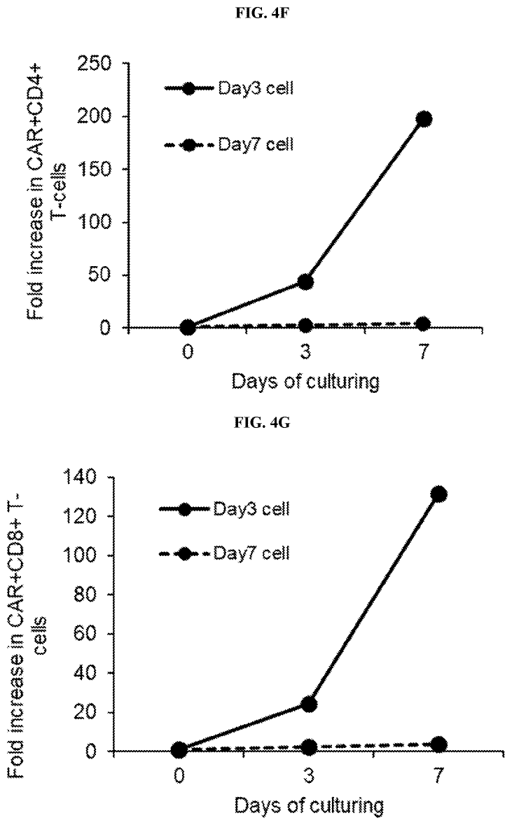

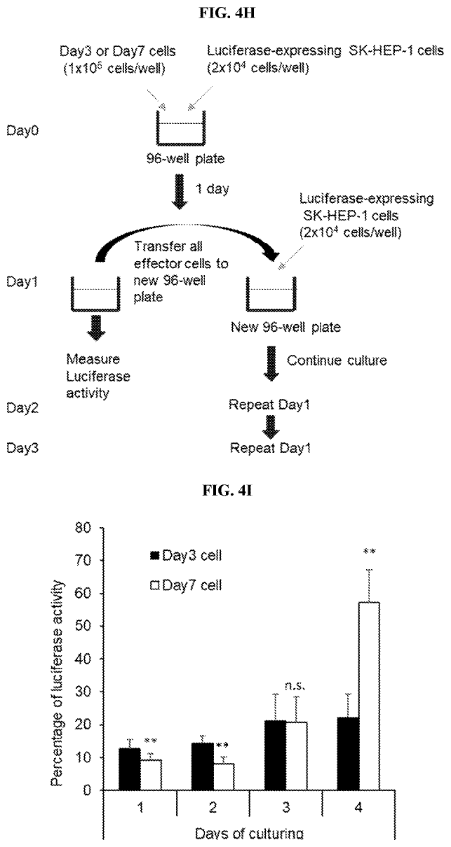

CAR-T cells expressing IL-7 gene and CCL19 gene were produced as described above (CAR described as SEQ ID NO: 2 was used). For preparation of Day 3 cells, expansion step was skipped (FIG. 4A). CAR protein expression which was measured by flowcytometry in Day3 cells was lower than in Day7 cells (FIG. 4B). On the other hand, CAR mRNA expression which was measured by qPCR as copy number in Day 3 cells was as same as in Day 7 cells (FIG. 4C). These indicated that transduction efficiency was not changed between Day 3 cells and Day 7 cells. The transition of T cell phenotype during CAR-T manufacturing was observed. At Day 2 to Day 3, Stem cell memory T-cells were peaked (FIG. 4D and FIG. 4E). Day 3 cells showed higher expandability (FIGS. 4F-4G) and cytotoxic capability (FIGS. 4H-4I) compared with Day 7 cells.

### Example 5: In Vivo Study with Day 3 and Day 7 CAR-T Cells

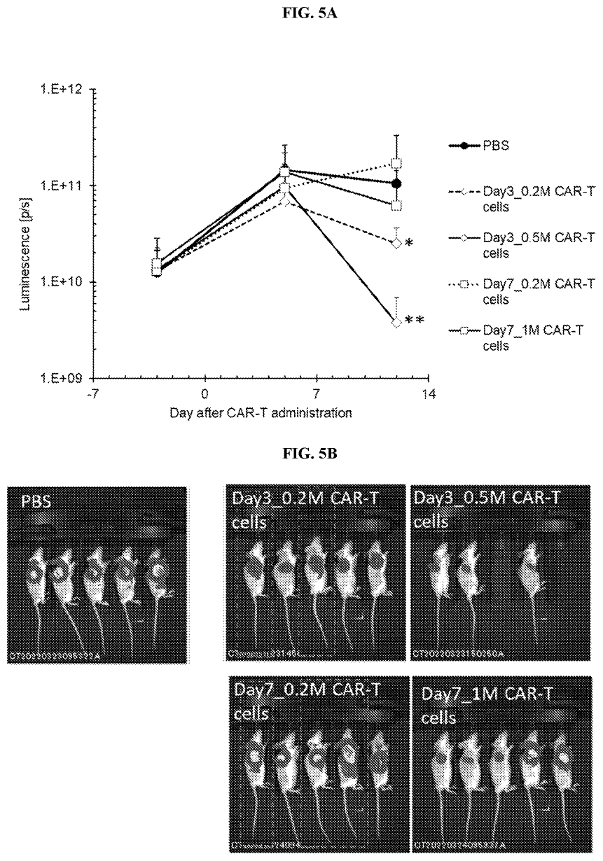

CAR-T cells produced in Example 4 were administrated after 7 days of GSU-LUC inoculation into NSG mice. 0.2 M and 0.5 M of Day3 cells (that produced without ex vivo expansion step) showed in vivo efficacy. However, 0.2 M and 1 M Day7 cells did not show it (FIGS. 5A and 5B).

### Example 6: In Vivo Study with Day 3 and Day 7 CAR-T Cells Manufactured in a Single Vessel by CliniMACS Prodigy

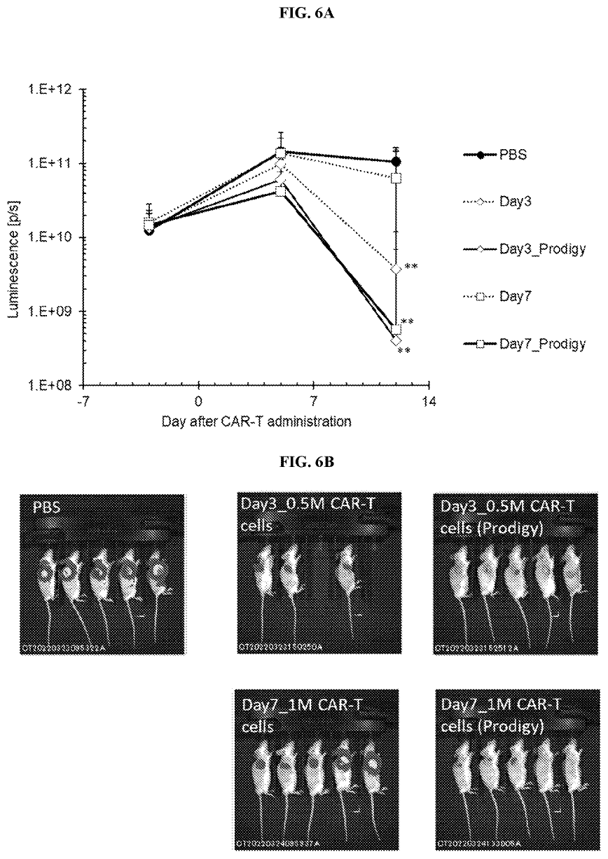

CAR-T cells expressing IL-7 gene and CCL19 gene were produced with or without CliniMACS prodigy (CAR described as SEQ ID NO: 2 was used). The efficacy of CAR-T cells which were manufactured in a single vessel with CliniMACS Prodigy showed tendency to strong compared with CAR-T cells which were manufactured without CliniMACS Prodigy (FIGS. 6A and 6B).

### Example 7: In Vitro Characterization of Day3 and Day7 CAR-T Cells (Unarmored)

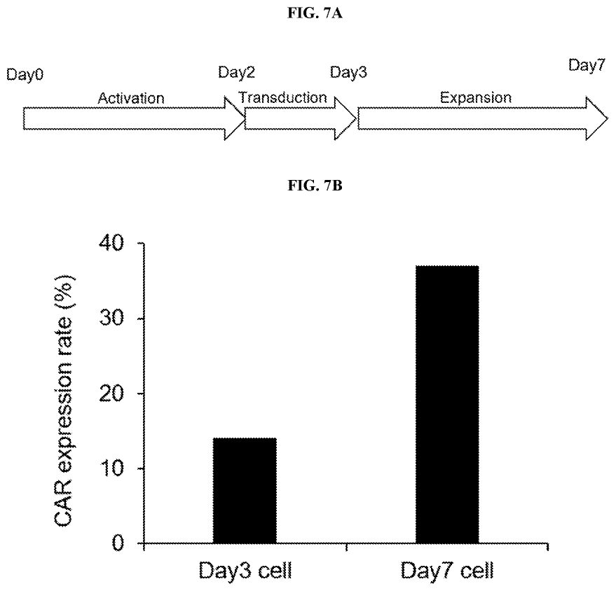

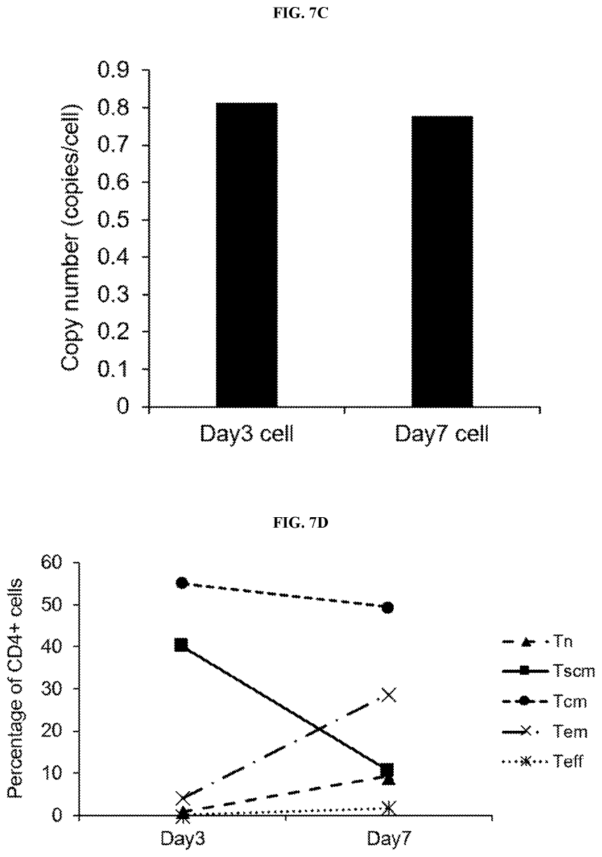

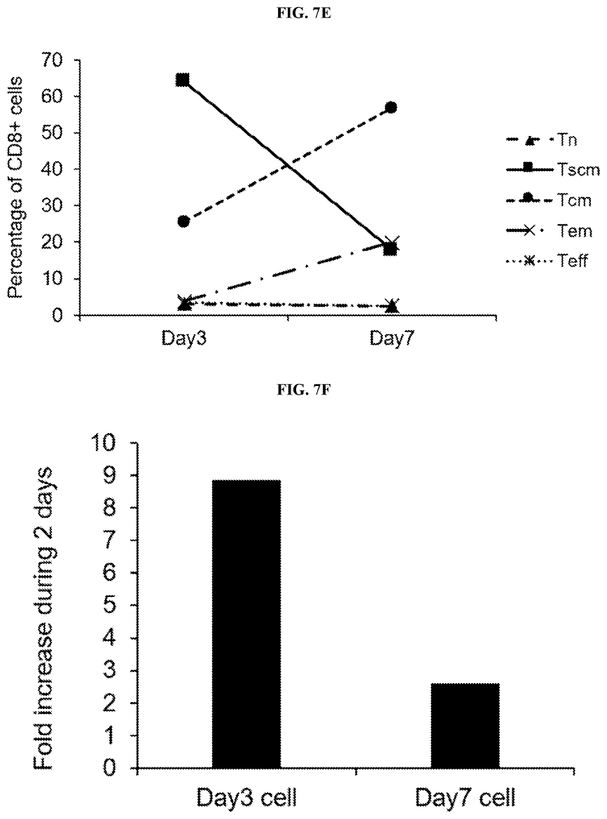

Unarmored CAR-T cells (not expressing exogenous cytokine or chemokine genes) were produced as described above (CAR described as SEQ ID NO: 2 was used). For preparation of Day 3 cells, the expansion step was skipped (FIG. 7A). CAR protein expression, which was measured by flowcytometry in Day 3 cells, was lower than in Day 7 cells (FIG. 7B). On the other hand, Day 3 cells CAR mRNA expression, which was measured by qPCR as copy number, was the same as in Day 7 cells (FIG. 7C). These results indicated that transduction efficiency was not changed between Day 3 cells and Day 7 cells. The T cell phenotype of Day 3 cells and Day 7 cells were observed. Day 3 cells showed a higher stem cell memory T-cells (Tscm) population compared with Day 7 cells (FIGS. 7D-7E). In addition, Day 3 cells showed higher expandability compared with Day 7 cells (FIG. 7F).

### Example 8: In Vivo Study with Day 3 and Day 7 CAR-T Cells (Unarmored)

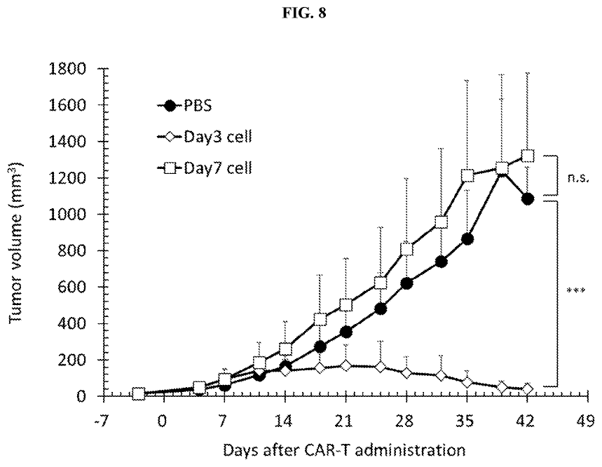

GSU cells were subcutaneously inoculated into NSG mice. Seven days after inoculation, either Day 3 and Day 7 unarmored CAR-T cells manufactured according to Example 7 in an amount of 1×10⁵, or PBS were intravenously administered to the mice. Day 3 unarmored CAR-T cells (that were produced without ex vivo expansion step) showed in vivo efficacy, but Day 7 unarmored CAR-T cells did not show in vivo efficacy (FIG. 8).

### Example 9: In Vitro Characterization of Day 3 and Day 7 CAR-T Cells Manufactured with CliniMACS Prodigy

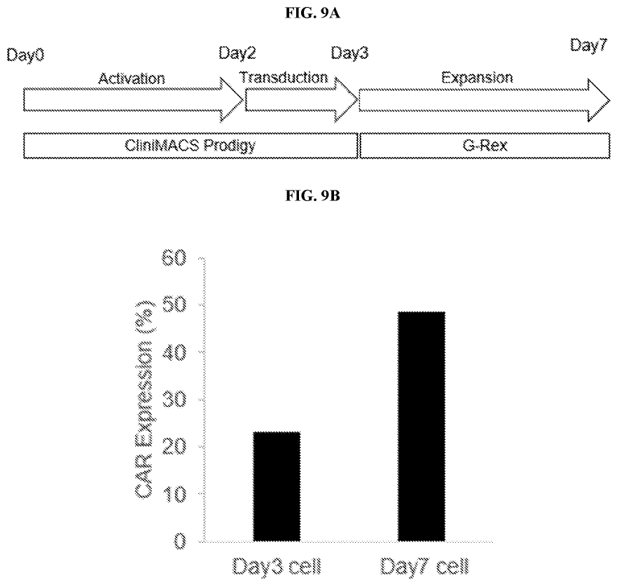

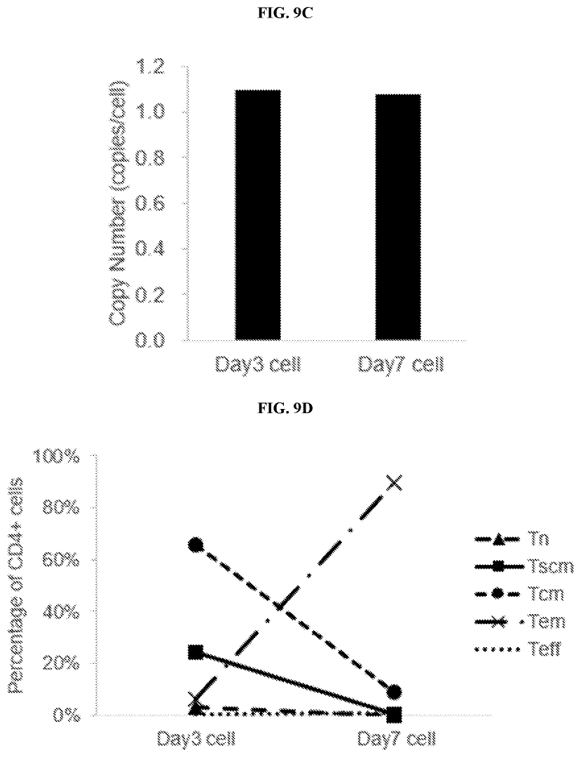

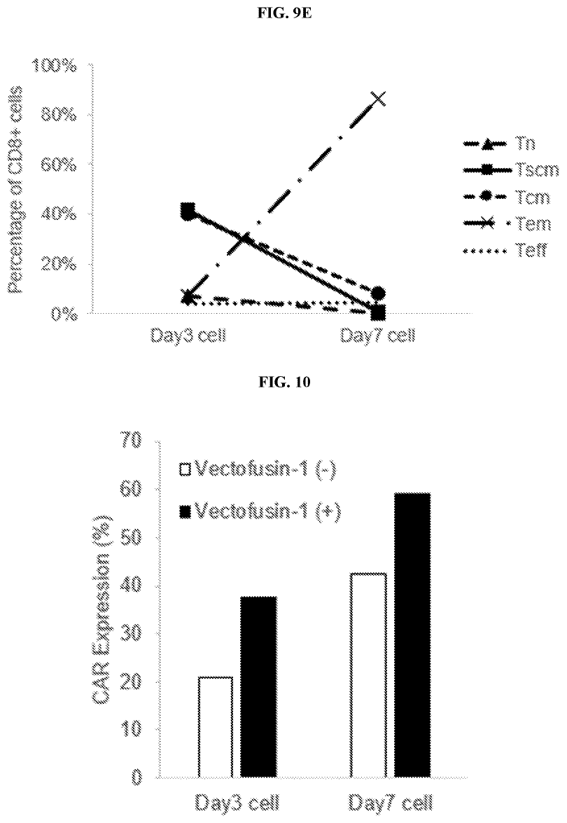

CAR-T cells expressing the IL-7 gene and CCL19 gene were produced with CliniMACS Prodigy according to the method “Production of CAR-T Cells with CliniMACS Prodigy” described in paragraph [00150]. CAR comprising the amino acid sequence of SEQ ID NO: 2 was used. FIG. 9A shows the scheme of the CAR-T cell manufacturing process. CAR-T cells were manufactured with CliniMACS Prodigy (Day 3 CAR-T cells). Day 3 CAR-T cells were further cultured in G-Rex culture bottles for 4 days (Day 7 CAR-T cells). FIGS. 9B-9C shows the results of the percentage of CAR-expressing cells (FIG. 9B) and the copy number in the cells (FIG. 9C). FIGS. 9D-9E show changes in T cell phenotype during manufacturing in CD4+ and CD8+ T cells, respectively. Day 3 cells showed a higher central memory and stem cell memory T-cells populations compared with Day 7 cells.

### Example 10: Day 3 and Day 7 CAR-T Cells Manufactured with CliniMACS Prodigy with or without Vectofusin-1 in Transduction Process

CAR-T cells expressing the IL-7 gene and CCL19 gene were produced with CliniMACS prodigy with or without Vectofusin-1 in the transduction step. CAR comprising the amino acid sequence of SEQ ID NO: 2 was used. FIG. 10 shows the result of the percentage of CAR-expressing cells manufactured with or without Vectofusion-1.

Those of ordinary skill in the art will recognize or be able to ascertain using no more than routine experimentation, many equivalents to the specific embodiments of the disclosure described herein. Such equivalents are intended to be encompassed by the following claims.

TABLE 1
SEQ ID NO. 1 (CAR)
QVQLVQSGAEVKKPGASVKVSCKASGYSFTGYNMNWVRQAPGQGLEWIG
NIDPYYGGTSYNQKFKGRATLTVDTSTSTAYMELRSLRSDDTAVYYCAR
GDYRAYYFDYWGQGTTVTVSSGGGGSGGGGSGGGGSDIQMTQSPSSLSA
SVGDRVTITCKASQNVRTAVAWYQQKPGKAPKALIYLASNRHTGVPSRF
SGSGSGTDFTLTISSLQPEDFATYYCLQHWNYPLTFGGGTKVEIKAAAF
VPVFLPAKPTTTPAPRPPTPAPTIASQPLSLRPEACRPAAGGAVHTRGL
DFACDIYIWAPLAGTCGVLLLSLVITLYCNHRNRSKRSRLLHSDYMNMT
PRRPGPTRKHYQPYAPPRDFAAYRSRFSVVKRGRKKLLYIFKQPFMRPV
QTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIG
MKGERRRGKGHDGLYQGLSTATKDTYDALHMQALPPR
SEQ ID NO. 2 (CAR)
MDWTWRILFLVAAATGAHSQVQLQQSGPGLVTPSQTLSLTCAISGDSVS
SNSATWNWIRQSPSRGLEWLGRTYYRSKWYNDYAVSVKSRMSINPDTSK
NQFSLQLNSVTPEDTAVYYCARGMMTYYYGMDVWGQGTTVTVSSGILGS
GGGGSGGGGSGGGGSQPVLTQSSSLSASPGASASLTCTLRSGINVGPYR
IYWYQQKPGSPPQYLLNYKSDSDKQQGSGVPSRFSGSKDASANAGVLLI
SGLRSEDEADYYCMIWHSSAAVFGGGTQLTVLSAAATTTPAPRPPTPAP
TIASQPLSLRPEACRPAAGGAVHTRGLDFACDIYIWAPLAGTCGVLLLS
LVITLYCKRGRKKLLYIFKQPFMRPVQTTQEEDGCSCRFPEEEEGGCEL
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
SEQ ID NO. 3 (IL7)
MFHVSFRYIFGLPPLILVLLPVASSDCDIEGKDGKQYESVLMVSIDQLL
DSMKEIGSNCLNNEFNFFKRHICDANKEGMFLFRAARKLRQFLKMNSTG
DFDLHLLKVSEGTTILLNCTGQVKGRKPAALGEAQPTKSLEENKSLKEQ
KKLNDLCFLKRLLQEIKTCWNKILMGTKEH
SEQ ID NO. 4 (CCL19)
MALLLALSLLVLWTSPAPTLSGTNDAEDCCLSVTQKPIPGYIVRNFHYL
LIKDGCRVPAVVFTTLRGRQLCAPPDQPWVERIIQRLQRTSAKMKRRSS

1. A method of producing a population of T cells that express an exogenous gene product, the method comprising:
(i) contacting a population of T cells with a stimulatory agent,
(ii) contacting the population of T cells with a retroviral vector that comprises a nucleic acid molecule encoding the exogenous gene product, thereby providing a population of T cells that express the exogenous gene, and
(iii) collecting the population of T cells expressing the exogenous gene product for storage or administration,
wherein:
the population of T cells expressing exogenous gene product from step (iii) are not expanded, or are expanded by no more than 200% as assessed by the number of living cells compared to the population of T cells at the beginning of step (i).

2. The method of claim 1, wherein the exogenous gene product is a chimeric antigen receptor (CAR).

3. The method of claim 1, wherein the stimulatory agent comprises a CD3 binding domain.

4. The method of claim 1, wherein the retroviral vector is a gamma retroviral vector.

5. The method of claim 3, wherein the gamma retroviral vector is selected from a pMSGV vector, a pMSCV vector, a pSFG vector, or a derivative thereof.

6. The method of claim 1, wherein steps (i)-(iii) are performed in a single vessel.

7. The method of claim 1, wherein step (ii) is performed in the presence of a soluble additive of a cationic amphipathic peptide.

8. The method of claim 1, wherein step (ii) is not initiated until after completion of step (i).

9. The method of claim 1, wherein, prior to step (i), the population of T cells is enriched for T cells that express CD3, CD4 and/or CD8.

10. The method of claim 1, wherein step (i) is performed in about 4 to about 96 hours.


SEQUENCE LISTING (4 sequences)

SEQ ID NO: 1 - protein, 527 aa, synthetic construct
QVQLVQSGAEVKKPGASVKVSCKASGYSFTGYNMNWVRQAPGQGLEWIGNIDPYYGGTSYNQKFKGRATLTVDTSTSTAYMELRSLRSDDTAVYYCARGDYRAYYFDYWGQGTTVTVSSGGGGSGGGGSGGGGSDIQMTQSPSSLSASVGDRVTITCKASQNVRTAVAWYQQKPGKAPKALIYLASNRHTGVPSRFSGSGSGTDFTLTISSLQPEDFATYYCLQHWNYPLTFGGGTKVEIKAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 504 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 4 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS